(19) United States
(12) Patent Application Publication
(10) Pub. No.: US 2003/0207811 A1
(43) Pub. Date: Nov. 6, 2003

(54) Method of treating retinopathy of prematurity using somatostatin analogs

(76) Inventors: Bruce K. Schrier, Wooster, OH (US); Rosemary D. Higgins, McLean, VA (US)

Correspondence Address: Nanda P.B.A. Kumar, 1650 Market Street, 2500 One Liberty Place, Philadelphia, PA, 19103, US

(21) Appl. No.: 10/138,554

(22) Filed: May 3, 2002

(51) Int. Cl. A61K 38/07

(52) U.S. Cl. 514/16

(57) ABSTRACT

The present invention discloses methods of treating or preventing retinopathy of prematurity in neonatal mammals involving the use of an amount of a somatostatin analog containing composition sufficient to provide a therapeutic benefit without affecting growth of the neonates.

### FIELD OF THE INVENTION

The present invention is directed to a method of preventing or inhibiting retinopathy of prematurity (ROP), with compounds which specifically or selectively exert their effects through somatostatin receptors. More specifically, the present invention is directed to the use of somatostatin analogs to prevent or inhibit ROP.

### BACKGROUND OF THE INVENTION

Retinopathy of prematurity (ROP), also called “retrolental fibroplasia”, is a serious bilateral ocular disorder of premature infants, particularly those whose birth weight is less than 1500 g. Approximately 80% of infants with birth weights below 1000 g develop ROP. The disorder is characterized by abnormal retinal vascularization that results from the following circumstance: Since normal human retinal vascularization begins at about mid-pregnancy and is complete at full term, prematurely born infants are born before this process is completed. Ex-uterine conditions may not be fully conducive to normal completion of retinal vascularization and other organ development. Particularly, pulmonary development is often inadequate in such pre-term infants to provide adequate oxygenation of developing tissues and organs. In many such instances, therefore, it is necessary to supplement the infant's oxygen supply to achieve survival and adequate development. It appears that abnormal vascularization of ROP is due to the initial relative hyperoxia followed by relative hypoxia subsequently experienced by the retina. It is not clear whether the abnormal vascularization of ROP is due to the oxygen directly or a result of the relative hypoxia experienced by the retina when the supplementation is stopped, but the pathology is characteristic of the proliferative (also called “ischemic”) retinopathies such as proliferative diabetic retinopathy (PDR) and age-related macular degeneration (ARMD), in addition to ROP. The proliferative retinopathies are characterized by excessive blood vessel growth (neovascularization or angiogenesis), hemorrhage, vascular leakage, scarring and sometimes retinal detachment. These retinal damages during early development may lead to myopia, strabismus, amblyopia, and risk of retinal detachments later in life. See, for example, Bossi et al., 1995, Intensive Care Med, 21: 241-6. Laser treatments may be used to limit neovascularization, but they contribute to scarring and would be better avoided when possible.

It is accepted in the art that the proliferative retinopathies result from ischemic injury to retinal vessels with subsequent compensatory angiogenesis. Increased growth hormone (GH) and insulin-like growth factor-1 (IGF-1) have been implicated in retinopathy. Diabetic patients with progressive retinopathy have been shown to have higher serum levels of GH and IGF-1. Poor glycemic control in the type I diabetic patient has been associated with increased serum IGFBP-1 and increased GH which can result in a reduction of IGF-1. IFGBPs can bind to IGF-1 by reducing the availability of IGF-1. IGF-1 has been postulated to have protective role in diabetic retinopathy (Janssen et al., 2000, Clin. Endocrinol. 52, 1-9).

Animal models have been developed to assist in understanding of these retinopathies. Oxygen-induced retinopathy (OIR) in neonatal mice has been developed as an animal model for ROP. The mouse is born at full term with highly immature retinal vascularization which matures during the first 3 weeks or so of postnatal growth. Thus, the neonatal mouse mimics the retinal development of the human fetus during the second half of gestation, and also, therefore, the prematurely born human infant. These models utilize treatment with oxygen during this development period to mimic the oxygen-induced ROP of humans. Using one such model, artisans in this field (Smith et al., 1994, Invest. Ophthalmol. Vis. Sci. 35, 101-11; Smith et al., 1997, Science 276, 1706-9; Smith, et al., 1999, Nat. Med. 5, 1390-5) have shown the important roles of GH, vascular endothelial growth factor (VEGF) and IGF-1 in the pathogenesis of this OIR. Recently, the relationship between VEGF and IGF-1 has been described in retinopathy of prematurity (Hellstrom et al., 2001, Proc. Natl. Acad. Sci. 98, 5804-8). When preterm birth occurs, IGF-1 levels fall below in utero levels and vessel growth ceases despite the presence of VEGF at the vessel front. Higher oxygen levels may also decrease VEGF levels causing further lack of vessel growth. As the preterm retina grows in thickness, vessel growth does not always occur concomitantly, and the retina may become hypoxic. With concurrent hypoxia, VEGF and IFG-1 levels increase and neovascularization occurs. An increase in serum GH levels in rats during a state of relative hypoxia has also been reported (Averbukh et al., 1998, Metabolism 47, 1331-6).

Somatostatin analogs have been proposed for inhibition or prevention of some of the ischemic retinopathies. Use of octreotide and lanreotide for PDR has been reported, the latter also for ARMD. In spite of a previous failure to demonstrate octreotide's efficacy (Grant et al., 1996, Ophthalmol Vis Sci 37: S958) in prevention of PDR, a more recent pilot study (Grant et al., 2000, Diab. Care 23, 504-9) suggested inhibition of progression by maximally tolerated doses of octreotide in patients in whom the euthyroid state was also maintained.

Specifically as to ROP, effect of various steroids and other drugs in prevention of OIR in a mouse model has also been investigated. Drugs such as diltiazem which is a calcium channel blocker (Higgins et al, 1999,. Curr. Eye Res. 18, 20-7), indomethacin which is an anti-inflammatory drug (Nandgaonkar et al., 1999), and dexamthasone which is a corticosteroid analog (Rotschild et al., 1999) all have been reported to have the capacity to prevent the effects of OIR.

The use of steroids and other potent drugs in therapy of ROP or OIR, is however, usually not without unpleasant or even serious side effects. For example, Dexamethasone has been used clinically to try to influence ROP, but with variable results—sometimes apparently detrimental. Use of corticosteroids in neonates as well as more mature individuals must be done with care because of the wide-ranging side effects of this class of compounds. While indomethacin is used in premature infants, it too is considered a toxic molecule that would not easily be used routinely to prevent ROP. Although diltiazem has been found to be effective in OIR model, this drug is also unlikely to be widely used in such fragile physiologies.

Therefore, a need exists in the prior art to develop drugs without unpleasant or even serious side effects and therefore of greater therapeutic benefit for the treatment of ROP. Some of the somatostatin analogs have been approved for human use, for example, for the treatment of acromegaly, for the suppression of severe diarrhea and flushing associated with malignant carcinoid syndrome, for the treatment of the profuse watery diarrhea associated with functional endocrine tumors. But the use of such analogs for prevention or treatment of ROP in humans or its animal models has not been known to date. Its use would be counter-intuitive if one considers that the premature infant is in desperate need of any GH and IGF-1 that it can generate. Somatostatin analogues, if they were to prevent ROP and would not retard growth, would be potentially a much desired and safer treatment for ROP than presently available pharmacological treatments.

### SUMMARY OF THE INVENTION

It has now been found that somatostatin analogs can be used to inhibit or prevent oxygen induced retinopathy without affecting growth of the treated neonatal mammal. Accordingly, the present invention provides, in general, methods of treating or preventing retinopathy of prematurity in neonatal mammals. The invention involves use of a pharmaceutical composition containing a somatostatin analog. Somatostatin analogs used in the present invention have increased serum half-lives in vivo relative to the natural somatostatin but mimic pharmacologic actions similar to the natural hormone.

In one aspect of the invention, somatostatin analogs used in the present invention are octreotide, a somatostatin analog having a serum half-life of octreotide, a somatostatin analog having a serum half-life more than that of octreotide, Woc4D and a somatostatin analog having a serum half-life of Woc4D.

In another aspect of the invention, the method uses somatostatin analogs having an ability to alter pituitary growth hormone expression (mRNA or the protein form) similar to that of the octreotide or the Woc-4D. The somatostatin analogs are capable of inhibiting GH expression in oxygen exposed mammals. The somatostatin analogs are capable of inhibiting GH expression in oxygen exposed mammals by at least 50%.

Specific somatostatin analogs for use in the present invention are octreotide, lanreotide (LANREOTIDE™), Vapreotide, Woc-2A, Woc-2B, Woc-3A, Woc-3B, Woc-4, Woc-4D, Woc-5 and Woc-8. In a preferred aspect, Octreotide is used at a dose of at least about 20 μg kg^{-1b} bid but not greater than 500 μg kg⁻¹ of birth weight.

In still another aspect of the invention, the method uses somatostatin analogs in a sustained or controlled release compositions such as octreotide acetate LAR (Sandostatin LAR®) and lanreotide (LANREOTIDE™) are used.

In yet another aspect of the invention, a pharmaceutical composition octreotide, lanreotide (LANREOTIDE™), Vapreotide, Woc-2A, Woc-2B, Woc-3A, Woc-3B, Woc-4, Woc-4D, Woc-5 or Woc-8 and a pharmaceutically acceptable carrier for treating or preventing retinopathy of prematurity in a neonatal mammal is used. The pharmaceutical composition may contain a pharmaceutically acceptable carrier in addition to the somatostatin analog. The pharmaceutically acceptable carrier can be water or a buffer solution. The composition is administered by a subcutaneous injection or continuous subcutaneous infusion. The composition is administered one, two or more times daily. The composition can be administered before, during, and/or after exposure of neonatal mammal to supplemental oxygen. The dose of a somatostatin analog should be sufficient to bring about suppression of retinopathy of prematurity (i.e., halt of ROP progression or regression of ROP). Maximally tolerated doses of a somatostatin analog can be administered. The administration of the drug may be repeated over a period of time.

### BRIEF DESCRIPTION OF THE DRAWINGS

FIG. 1 is a histogram showing median total retinopathy score for somatostatin analog, Woc4D or octreotide treatment.

FIG. 2 is a histogram showing pituitary growth hormone expression in various treatment groups.

FIG. 3 shows a composite photograph of fluorescein perfused retinal whole mounts (4-5× magnification). The retinal mounts are from room air reared (A), oxygen treated (B), oxygen and octreotide treated (C), and Woc4D treated (D) animals.

### DETAILED DESCRIPTION OF THE INVENTION

The present invention discloses methods for treating or preventing retinopathy of prematurity. The invention involves administration of analogs of somatostatin to achieve the desired effect in neonates having retinopathy or susceptible to retinopathy.

Somatostatin is a naturally-occurring peptide that antagonizes the release of GH from the pituitary gland. Therefore, somatostatin can be useful in control of excessive levels of GH, no matter the disorder which has caused such an excess. Its chemical structure is known in the art (see, for example, U.S. Pat. No. 5,876,761; Burgus et al., 1973, Proc. Nat. Acad. Sci. USA 70:684; McQuillan (ed.), 1980, Somatostatin, Vol. 2, Eden Press, Quebec, pp 1-238). It has been reported in the art that native somatostatin exerts its effects through as many as five different receptors designated SSTR1-5. The half-life of the native hormone in the blood stream is only about 1-3 minutes. In fact, the half-life of native somatostatin is so brief in the blood stream that it's use for anything beyond the control of acute esophageal bleeding has been found impractical.

The drugs of use in the methods of the present invention are preferably compositions containing somatostatin analogs rather than the natural somatostatin for the reasons apparent from the description below.

Somatostatin analogs, by comparison, are much more stable, have longer blood half-lives and consequently much longer acting. Peptide analogs of somatostatin that have longer half-lives have therefore been developed, and three of these are being used clinically: octreotide, lanreotide, and vapreotide. The primary use of these analogs has been for acromegaly, the disease that most prominently displays the effects of GH excess and its control. The analogs usually control very effectively the excessive secretion of GH and the attendant sequellae in acromegalics.

As is well known to those skilled in the art, a variety of other somatostatin analogs have been in existence for a long time. See, for example, the somatostatin analogs described in US patents having the following U.S. Pat. Nos. 4,904,642; 4,871,717; 4,853,371; 4,725,577; 4,684,620; 4,650,787; 4,603,120; 4,585,755; 4,522,813; 4,486,415; 4,485,101; 4,435,385; 4,395,403; 4,369,179; 4,360,516; 4,358,439; 4,328,214; 4,316,890; 4,310,518; 4,291,022; 4,238,481; 4,235,886; 4,224,190; 4,211,693; 4,190,648; 4,146,612; 4,133,782; see also the following: Van Binst et al., Peptide Res., 5:8 (1992); Prevost et al., Cancer Res., 52:893 (1992); Bachem California 1993-1994 Catalog 94-95 (1993), Woltering et al., 1991, J. Surg. Res. 50, 245-51; Woltering et al., 1999, J. Pept. Res. 53, 201-13; teachings of these patents and references are incorporated herein by reference.

Somatostatin analogs as used herein are those analogs which differ from the native hormone in their type and/or sequence of amino acids, but have in common their interaction with one or more types of the somatostatin receptors, or have pharmacologic properties mimicking those of the native hormone and have increased blood half-lives in vivo. The direct correlation between somatostatin receptor binding and inhibition of GH release has been reported (Rayner, 1993, Mol. Pharmacol., 43:838-44). Assays to test the ability of somatostatin analogs to inhibit release of GH are known (see, for example, U.S. Pat. No. 5,597,894).

For example, a number of somatostatin analogs have been produced by elimination of amino acids that are not absolutely required for activity and/or substitution of the native L-amino acids with the corresponding D-amino acid isomers. Some of these analogs not only have increased half-lives in vivo relative to the native somatostatin and hence are longer acting but are more potent receptor agonists than the native hormone. For example, the synthetic somatostatin analog octreotide acetate, which has the amino acid sequence D-Phe-Cys-Phe-D-Trp-Lys-Thr-Cys Thr(ol) (SEQ ID NO:1), has been reported to have a half-live of 1.7 hours and to be 45 to 70 times more potent clinically than native somatostatin in inhibition of growth hormone release. Octreotide is also effective in stimulating SSTR2 to the virtual exclusion of the other receptors, and this interaction is thought to be the basis of its activity in the many disease states in which it is being employed. LANREOTIDE™, an octapeptide somatostatin analog having the amino acid sequence Dβ-Nal-Cys-Tyr-D-Trp-Lys-Val-Cys-Thr(NH₂) (SEQ ID NO:2), has been reported to be 20 to 50 times more potent clinically than native somatostatin.

Accordingly, somatostatin analogs having a serum half-life greater native somatostatin can be made and used. Particularly, those somatostatin analogs having the efficacies of providing control of excessive growth hormone secretion in acromegalics, of symptoms of the carcinoid syndrome in patients with metastatic carcinoid, and of secretory diarrhea in patients with functional endocrine tumors (e.g. VIPomas, insulinomas, gastrinomas, glucagonomas, carcinoids, and the like) may be used in the methods of the present invention. Some of the examples of somatostatin analogs known to have these efficacies are octreotide, lanreotide (LANREOTIDE™) and Vapreotide.

Preferred known somatostatin analogs are octreotide, lanreotide (LANREOTIDE™), Vapreotide, Woc-2A, Woc-2B, Woc-3A, Woc-3B, Woc-4, Woc-4D, Woc-5 and Woc-8. Particularly preferred analogs are octreotide and Woc-4D. These preferred somatostatin analogs can be readily prepared according to the procedures known in the art. See, for example, the procedures disclosed in U.S. Pat. No. 5,411,943, U.S. Pat. No. 5,597,894 and the U.S. Pat. No. 6,346,601, the teachings of which are incorporated herein by reference.

Still preferred somatostatin analogs are those having a serum half-life of octreotide, those having a serum half-life more than that of octreotide, those having a serum half-life of Woc4D or those having an ability to alter pituitary growth hormone expression similar to that of octreotide or Woc4D.

The analogs can also be radiolabeled. For example, the analog Woc4D with N-terminal tyrosine residues is designed to be used for radioactive imaging. It is an agonist analog that exhibits high affinity interaction with SSTR5 as well as SSTR2. Applicant has found that the blood half-life of Woc4D in rats and primates is approximately 5 times longer than that of octreotide. Because of the longer half-life, Woc4D can be administered less frequently than the other known analogs with shorter half-lives in order to maintain therapeutic blood levels, thus further reducing morbidity and potential side effects. The method of treatment of the invention can be implemented as a preventive intervention when oxygen supplementation is to be used or as a therapy after the first signs of ROP have appeared.

Somatostatin analogs used in the present invention include either a free form or a pharmaceutically-acceptable salt form or in the form of complexes thereof. The acetate salt and pamoate salt are preferred salt forms. For example, octreotide acetate (Sandostatin®) or Woc-4D acetate or octreotide pamoate or Woc-4D pamoate can be used.

Guidance for dosages appropriate for practicing the methods of also controlled-release formulations of 10-14 days or longer of the invention can be based upon dosages recommended for somatostatin analogs already in the world market. Briefly, the dosage of active ingredient in the compositions for practicing this invention can vary according to a variety of factors including the biological activity and half-life of the analog selected, the type of administration or delivery of the drug, and patient dependent variables such as size, weight, and age and other factors appreciated by one skilled in the art.

Generally, dosage levels of between 25 μg/kg/day to 5 mg/kg/day of body weight daily are administered either as a single dose or divided into multiple doses to humans and other animals, e.g., mammals, to obtain the desired therapeutic effect. The specific amount administered is generally from about 1 μg to 1,000 or from about 200 μg to 5,000 μg, preferably about 20 μg to 500 μg, more preferably about 40 μg to 200 μg, normally about 100-500 μg/kg/day of body weight. The composition may be administered as a single dose or divided into multiple doses. The amount administered can be increased or decreased depending on the potency of the selected analog administere. The selected dose is such that it is sufficient to ameliorate ROP without causing the toxic effect such as GI toxicity. The potency of drug can be determined by GH levels, IGF-1, retinopathy scores or such other parameters known in the art.

Dosage amounts for some somatostatin analogs such as, for example, Woc-4D (a multi-tyrosinated analog), are generally lower than those required for other conventional somatostatin analogs due to the relative affinity and/or avidity of the multi-tyrosinated analogs for somatostatin receptors, and/or the enhanced half-life of such analogs known in the art. Somatostatin analogs for administration as therapeutic agents can be unlabeled, halogenated, fluorescinated, or radiolabeled. Where a radiolabeled analog is used, the peptide can be labeled just prior to administration, e.g., 24 hr or less before administration.

The modifier “about” is used herein to indicate certain preferred material amounts or ranges, which are not fixedly determined. The meaning will often be apparent to one of ordinary skill in the art. For Example, a recitation of 50 μg kg⁻¹ day⁻¹ of Woc4D or 20 μg kg⁻¹ bid of octreotide in reference to, for example, suppression of OIR would be interpreted to include other like dosages which can be expected to favor a useful effect or suppression of ROP such as, for example, 40 μg kg⁻¹ day⁻¹ or 60 μg kg⁻¹ day⁻¹ of Woc4D (15 μg kg⁻¹ bid or 30 μg kg⁻¹ bid of octreotide).

The somatostatin analogs can be administered by any conventional route, preferably by subcutaneous injection in the form of an injectable solution or suspension. The analogs can also be administered by infusion (see, Grant et al., 2000, Diabetes care 23:504-509), e.g., subcutaneous infusion or an intravenous infusion. Somatostatin analogs can also be administered intravitreous, intramuscular, intraarterial or intravenous, topical, targeted delivery, direct injection into the affected regions in the eye(s) or other routes known to one of ordinary skill in the art.

Further, a compsoition administered in the methods of the present invention can be in an immediate release form or a sustained or controlled release formulation. Immediate release forms do not have biodegradable polymers as part of the composition. Example is octreotide acetate in an acetate buffer. Controlled release forms have biodegradable polymers as part of the compositions. Such controlled release compositions are known in the art. Among those formulations, 10-19 days or longer controlled release formulations may be used. One such formulation is octreotide acetate LAR (Sandostatin LAR®) can be used (see, PDR 2000). The U.S. Pat. No. 5,672,659 teaches sustained release compositions comprising lanreotide (LANREOTIDE™) and a polyester. U.S. Pat. No. 5,595,760 teaches sustained release compositions comprising Lanreotide (LANREOTIDE™) in a gelable form. Other polymeric sustained release compositions comprising Lanreotide (LANREOTIDE™) and chitosan or Lanreotide (LANREOTIDE™) and cyclodextrin are also known in the art. See, also U.S. Pat. No. 5,688,530. The contents of the foregoing patents are incorporated herein by reference. For example, an injection containing a somatostatin analog 30 mg in a special (microparticulate) form can be administered from which it is very slowly released.

The use of immediate or of sustained release composition forms depends on the type of indications aimed at. If the indication consists of an acute or over-acute disorder, a treatment with an immediate form may be preferred over the same with a prolonged release composition. On the contrary, for preventive or long-term treatments, a prolonged release composition may generally be preferred.

The pharmaceutical compositions of the invention can contain compounds in addition to the somatostatin analogs and pharmaceutically acceptable carriers. For example, the commercially available somatostatin analog, octreotide acetate LAR (Sandostatin LAR ®), contains the following: octreotide acetate, D,L-lactic and glycolic acids copolymer, mannitol (and the diluent containing carboxymethylcellulose sodium, mannitol and water).

For example, a number of drugs in the form of eye drops for topical application have been developed. A solution containing chlorobutanol (0.55%), mannitol (1.2%) boric acid (0.6%) and exsiccated sodium phosphate (0.026%) is used as a carrier solution and/or as a diluent for an eye drug, PHOSPHOLINE IODIDE. Such pharmaceutically acceptable carriers can be used for topical application. Other pharmaceutically acceptable carriers or excipients that are known to enhance membrane permeability and cellular uptake of the drug can also be used for application to the eye. Such carriers are known to one skilled in the art.

Further, for example, the pharmaceutical composition can contain compounds to provide relief of symptoms associated with the condition to be treated (e.g., thyroxine or synthroid to ensure clinical euthyroid status in the patients, pain-relieving compounds, anti-inflammatories, etc.).

As discussed above, a mouse model of OIR is a standard experimental animal model for ROP in humans. Thus, one practicing the invention can readily use an OIR in mouse as an in vivo model system to demonstrate efficacy and relations potency for each selected somatostatin analog sufficient to treat or prevent before indicating the analog for human use. The effectiveness of somatostatin analogs in the new medical use for treating ROP disease can be better understood through the results of tests in OIR model of mouse described further in the example below.

### EXAMPLE

The example provided herein is carried out using standard laboratory techniques that are well known and routine to those of skill in the art, except where otherwise described in detail. The example is illustrative, but does not limit the invention.

The effect of two somatostatin analogs Woc4D and octreotide, on oxygen induced retinopathy in the mouse is demonstrated herein. Oxygen induced retinopathy was produced in C57BL6 mice. Octreotide and Woc4D were administered from post-natal day 12-16 (P12-P16). Retinopathy was assessed by a retinal scoring systems utilizing fluorescein perfused retinal whole mounts. Animals treated with Woc4D and octreoride respectively, had median retinopathy scores of 4(3,5) [median(25th, 75^{th} quartile)] with P=0.01 and 3.5(2.9, 4.3) with P=0.01 compared to oxygen and sham treated oxygen animals with scores of 6.6(5.3, 8.5) and 7.4(5.8, 8.6), respectively. Woc4D and octreotide treated animals had decreased blood vessel tufts and decreased extra-retinal neovascularization when compared to oxygen treated animals. Pituitary growth hormone (GH) mRNA expression was increased 8.3 fold by Woc4D treatment and 106 fold by oxygen exposure, and oxygen stimulated GH and mRNA was markedly reduced by Woc4D as well as octreotide. Growth as measured by animal weight was unaffected by either treatment. Woc4D and octreotide inhibited retinal neovascularization in an equally effective manner in the mouse model of oxygen induced retinopathy. The details of various aspects of this study are described below:

Animal Model of Oxygen Induced Retinopathy

This protocol was approved by the Georgetown University Animal Care and Use committee and adhered to the Association for Research in Vision and Ophthalmology Statement for the Use of Animals in Ophthalmic and Vision Research. C57BL6 mice were obtained from Taconic Laboratories (Germantown, N.Y.). Neonatal mice were placed in an infant incubator with their nursing mother with 75% oxygen at P7 and remained in oxygen until removal to room air at P12 (Smith et al., 1994 Invest. Ophthalmol. Vis. Sci. 35, 101-11). Oxygen was delivered at 75±2%, measured with an oxygen analyser (Hudson Ventonics, Temecula, Calif., U.S.A.), and checked at least twice daily. Individual litters were either oxygen or room air reared. Within litters, differential treatment was performed including no treatment, octreotide, Woc4D, and sham injection. Animals were treated with injections for 5 days from P12 to P16. Animal killing was performed using a lethal dose of pentobarbital (120 mg/kg i.p.). Animals were perfused with fluorescein conjugated dextran as previously described (D'Amato et al., 1993, Microvasc. Res. 46, 135-42) and retinopathy was assessed by a retinal scoring system shown in Table I.

Administration of Woc4D and Octreotide

Woc4D was synthesized by California Peptide research with following sequence: Dtyr-Dtyr-Dtyr-Dtyr-cyclo(Cys-Phe-DTrp-Lys-Thr-Cys)-Thr-NH₂ (SEQ ID NO: 3). It was received and used as the acetate salt. Octreotide acetate was purchased from Polypeptide Laboratories. Woc4D was initially used at 20 μg kg⁻¹ day⁻¹ in a single injection subcutaneously and octreotide was used at 10 μg kg⁻¹ dose⁻¹ given twice daily subcutaneously from P12-P16. The doses were increased to 50 μg kg⁻¹ day⁻¹ of Woc4D and 20 μg kg⁻¹ bid of octreotide following initial experiments. The analogs were dissolved in 33 mM acetate buffer (pH 5) with 135 mM NaCl. Sham injections were performed with that vehicle.

Growth Hormone Reverse Transcriptase Polymerase Chain Reaction

In order to assess the effect of oxygen, octreotide, and Woc4D on growth hormone expression, pituitary tissue was obtained for GH expression. Three to four animals per group (room air, room air+Woc4D, room air+octreotide, oxygen, oxygen+Woc4D and oxygen+octreotide) were killed on day 16 following a 5 day treatment (P12-P 16) with no drug, octreotide (20 μg kg⁻¹ bid) or Woc4D (50 μg kg⁻¹ day⁻¹) and pituitary glands were obtained from them. RNA was extracted using TRIzol reagent (Life Technologies, Rockville, Md., U.S.A.) as described by the manufacturer and reverse transcriptase polymerase chain reaction (RT-PCR) was performed. Ten μg of RNA and 2 μM oligo(dT) 16 (total volume 22 μl) were heated at 68° C. for 2 min and cooled on ice. First strand synthesis was performed by incubating the RNA and oligo(dT) in a reaction mixture (total volume, 50 μl) containing 50 mM Tris-HCl, pH 8.5, 40 mM KCl, 8 mM MgCl₂, 2 mM DTT, 50 U reverse transcriptase, and 0.8 mM each of dATP, dCTP, dGTP, and dTTP. The mixture was incubated at 42° C. for 1 hour, then 99° C. for 5 min. The resultant cDNA was diluted with 100 μl of water and stored at −20° C. until PCR was performed. PCR reaction mixture (25% total volume) was prepared with 0.2 mM each of dATP, dCTP, dGTP, and dTTP; 50 mM KCl; 10 mM Tris-HCl, pH 8.3; 1.5 mM MgCl₂; 100 ng each of the mouse GH forward and reverse primers (see sequence below, Life Technologies, Rockville, Md., U.S.A.); 0.625 U of Taq DNA polymerase (Perkin-Elmer, Branchburg, N.J., U.S.A.); and 3 μl of the diluted cDNA. The mixture was incubated for 4 min at 94° C., followed by 28 cycles of 45 s at 94° C., 45 s at 58° C., and 45 s at 72° C., followed by 7 min at 72° C., in a PCR apparatus (model 2400, Perkin Elmer). To verify equal amounts of RNA were used in the PCR reaction and to verify uniform amplification process, β-actin mRNA was also amplified from the sample as an internal control. PCR products were separated on a 1.2% agarose gel and were visualized by staining with ethidium bromide. A 100 bp DNA ladder was used as a size marker. The number of cycles versus intensity of the PCR band was evaluated to determine the optimum number of cycles to be in the linear range. Gels were photographed and scanned for density using the Quantiscan program (Biosoft, Ferguson, Mo., U.S.A.). The RT-PCR was repeated three times for the experiment. The PCR products were sequenced for confirmation. The oligonucleotide primer sets used to amplify GH (Linzer et a., 1985, J. Biol. Chem. 260, 9574-9) and β-actin (R&D Systems, Minneapolis, Minn., U.S.A.) were: (forward) 5′CTGCTGACACCTACAAAGAG3′ (SEQ ID NO: 4) and (reverse) 5′GCGTCAAACTTGTCATAGG3′ (SEQ ID NO: 5); and (forward) 5′-CTACAATGAGCTGCGTGTGG-3′ (SEQ ID NO: 6) and (reverse) 5′-AAGGAAGGCTGGAAGAGTGC-3′ (SEQ ID NO: 7). The resultant PCR products were 385 bp (GH) and 528 bp (β-actin).

Statistical Analyses

Retinopathy scores were evaluated by the Kruskal-Wallis test for the overall group and by the Mann-Whitney test to determine differences between groups. Animals weights and GH expression were evaluated using Student's t-test. Statistical significance was determined at the P<0.05 level.

Analysis of the In Vivo Effects of Somatostatin Analogs

Initial studies were performed using Woc4D at does of 20 μg kg⁻¹ day⁻¹ from P12 to P16 octreotide at a dose of 10 μg kg⁻¹ bid from P12 to P16. Woc4D treated animals had a median total retinopathy score of 6(3,7); octreotide treated animals also had score of 6(3,7). Oxygen reared animals had a score of 7(5,8). Based on these results doses were adjusted upwards.

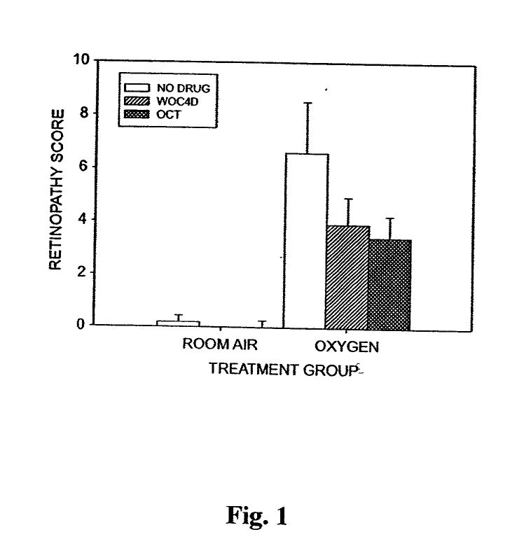

Median total retinopathy score for Woc4D and octreotide treatments are showin in FIG. 1. The number of animals in each group were as follows: room air, no drug, n=11; room air, Woc4D, n=7; room air, octreotide, n=7; oxygen, no drug, n=20; oxygen, Woc4D, n=11; and oxygen, octreotide, n=12; Sham treated animals had retinopathy scores of 0.375(0, 0.5) in the room air reared group and 7.375(5.8, 8.6) in the oxygen group. The error bar denotes 75th quartile for Woc4D and octreotide treatments. By Kruskal Wallis test P<0.01 for entire group; by Mann-Whitney test, P=0.01 for oxygen versus control and oxygen versus oxygen+Woc4D; and by Mann-Whitney test, P=0.01 for oxygen versus control and oxygen versus+octreotide. Animals treated with higher concentrations of Woc4D (50 μg kg⁻¹ day⁻¹) had a significant improvement in their retinopathy scores when compared to oxygen and oxygen and sham treated animals as shown in FIG. 1. Woc4D improved retinopathy (P=0.01) when compared to oxygen alone. Pups treated with higher dose of octreotide (20 μg kg⁻¹ bid) also had a significant improvement in their retinopathy scores as shown in FIG. 1 (P=0.01). Specifically, Woc4D and octreotide provided improvement in blood vessel tufts and in extra-retinal neovascularization as shown in Table II.

Because the analogs may have their effect in part, by suppression of serum GH levels and subsequent reduction of serum IGF-1 levels, pituitary GH mRNA expression and growth were examined. Weight data for various treatment groups are shown in Table III. Surprisingly, there was no effect on growth by any of the treatment regiments and the use of somatostatin analogs to control retinopathy in the mouse model was not detrimental to overall growth rate.

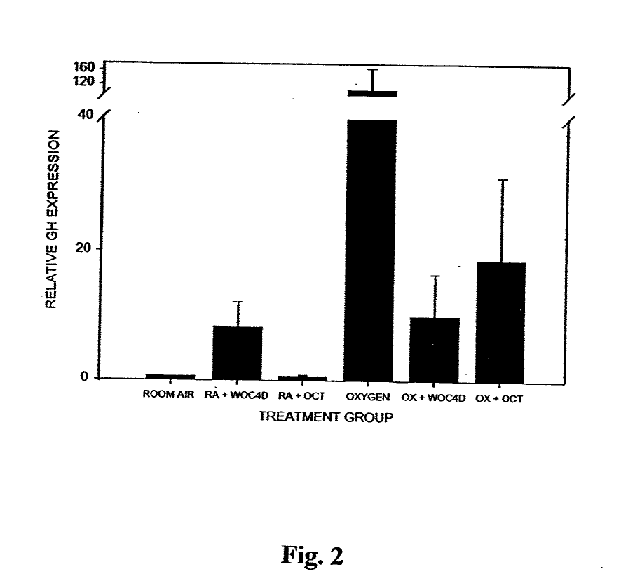

Shown in FIG. 2 is pituitary growth hormone mRNA expression data for each treatment group. Relative GH expression is shown on the y-axis and the various treatment groups are shown on the x-axis. Values are expressed as mean±S.E.M., with room air being normalized to one. GH mRNA expression as measured by RT-PCR was increased 8.3-fold (4-16-fold range) by Woc4D treatment in room air reared animals and increased 106-fold (27-227-fold range) by oxygen exposure. It should be noted that this altered pituitary GH expression did not translate to an effect on growth rate per se.

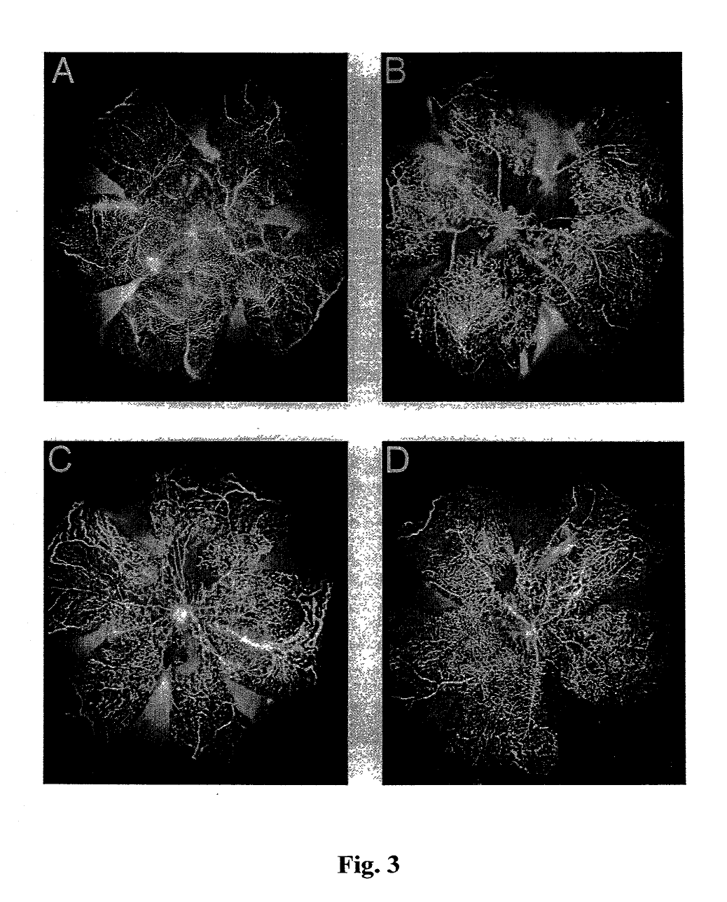

FIG. 3 shows a composite photograph of representative retinal whole mounts, (A) depicts a fluorescein perfused retina from a room air reared animal. (B) shows an oxygen treated retina. Note the central loss of blood vessels and blood vessel tufts. (C) shows a retinal whole mount from an octreotide treated animal. Similarly, (D) shows a retina from a Woc4D treated animal with less retinal vascular pathology. (B-D) show larger, more engorged vessels characteristic of oxygen exposed retinas. 4.5× magnification.

All publications and references, including but not limited to patent applications, cited in this specification, are herein incorporated by reference in their entirety as if each individual publication or reference were specifically and individually indicated to be incorporated by reference herein as being fully set forth. While this invention has been described with reference to specific embodiments, those of ordinary skill in the art will understand that variations in these methods and compositions may be used and that it is intended that the invention may be practiced otherwise than as specifically described herein. Accordingly, this invention includes all modifications encompassed within the spirit and scope of the invention as defined by the claims.

TABLE I
Retinopathy scoring system

| | Score | | | | |
|---|---|---|---|---|---|
| Parameter | 0 | 1 | 2 | 3 | 4 |
| Blood Vessel | Complete | Incomplete | Incomplete | Incomplete | |
| growth | | outer third | middle third | inner third | |
| Blood vessel | None | Few, scattered | 3-5 clock | 6-8 clock | 9-12 clock |
| tufts | | <3 clock hours | hours | hours | hours |
| Extra retinal | None | Mild <3 | Moderate 3-6 | Severe >6 | |
| neovascularization | | clock hours | clock hours | clock hours | |
| Central | None | Mild early | Moderate | Severe extending | |
| vasoconstriction | | zone 1 (inner | throughout | to zone 2 | |
| | | 50% of zone 1) | zone 1 (outer | | |
| | | | 50% of zone 1) | | |
| Retinal hemorrhage | Absent | Present | Moderate | Severe | |
| Blood vessel | None | Mild <3 | 3-6 clock | >6 clock | |
| tortuosity | | clock hours | hours | hours | |

TABLE II
Retinopathy subscores [expressed as median (25^{th}, 75^{th} quartile); oct, octreotide]

| | Room | Room air + | Room air + | | | Oxygen + | | Oxygen + oct | |
|---|---|---|---|---|---|---|---|---|---|
| | air | Woc4D 50 | oct 20 bid | Oxygen | | Woc4D 50 | | 20 bid | |
| Number of animals (n) | 11 | 7 | 7 | 20 | | 11 | | 12 | |
| Blood vessel growth | 0 (0, 0) | 0 (0, 0) | 0 (0, 0) | 0 · 25 | (0, 0 · 5) | 0 | (0, 0 · 375) | 0 | (0, 0) |
| Blood vessel tufts | 0 (0, 0) | 0 (0, 0) | 0 (0, 0) | 1 · 25 | (0.69, 2 · 3) | 0 · 5 | (0 · 5, 0 · 63)* | 0 · 5 | (0, 1)* |
| Extra retinal | 0 (0, 0) | 0 (0, 0) | 0 (0, 0) | 0 · 75 | (0 · 5,1) | 0 | (0, 0 · 125)** | 0 · 125 | (0, 05)** |
| neovascularization | | | | | | | | | |
| Central | 0 (0, 0) | 0 (0, 0) | 0 (0, 0) | 1 · 88 | (1, 2 · 5) | 1 · 5 | (1 · 125, 2 · 25) | 1 | (1, 1 · 25) |
| vasoconstriction | | | | | | | | | |
| Hemorrhage | 0 (0, 0) | 0 (0, 0) | 0 (0, 0) | 1 | (0 · 5, 1) | 1 | (0 · 5, 1) | 0 · 5 | (0 · 38, 0 · 63) |
| Blood vessel tortuosity | 0 (0, 0) | 0 (0, 0) | 0 (0, 0) | 1 · 5 | (1, 1 · 18) | 1 | (0 · 75, 1 · 38) | 1 · 13 | (0 · 75, 1 · 5) |

*Denotes P < 0 · 05 when compared to oxygen treated group.
**Denotes P < 0 · 001 when compared to oxygen treated group.

TABLE III
Weight data for various treatment groups

| | | | | | Weight change |
|---|---|---|---|---|---|
| | Number of | | | | P12-Psac |
| | animals (n) | P7 Weight | P12 Weight | PSAC Weight | (g day⁻¹) |
| Room air | 13 | 4 · 03 ± 0 · 69 | 6 · 44 ± 0 · 98 | 8 · 59 ± 0 · 86 | 0 · 33 ± 0 · 05 |
| Room air + Woc4D 20 μg | 2 | 4 · 02 ± 0 · 66 | 6 · 76 ± 0 · 77 | 9 · 08 ± 1 · 07 | 0 · 29 ± 0 · 04 |
| Room air + oct 10 μg bid | 4 | 4 · 25 ± 0 · 74 | 7 · 02 ± 0 · 67 | 8 · 69 ± 0 · 39 | 0 · 30 ± 0 · 07 |
| Room air + Woc4d 50 μg | 7 | 4 · 10 ± 0 · 39 | 6 · 54 ± 0 · 38 | 8 · 64 ± 0 · 57 | 0 · 35 ± 0 · 04 |
| Room air + oct 20 μg bid | 7 | 4 · 08 ± 0 · 12 | 6 · 47 ± 0 · 59 | 8 · 83 ± 0 · 71 | 0 · 37 ± 0 · 05 |
| Room air sham | 5 | 4 · 42 ± 0 · 40 | 7 · 25 ± 0 · 61 | 8 · 94 ± 0 · 61 | 0 · 29 ± 0 · 08 |
| Oxygen | 21 | 3 · 81 ± 0 · 92 | 5 · 91 ± 1 · 15 | 8 · 12 ± 1 · 36 | 0 · 31 ± 0 · 44 |
| Oxygen + Woc4D 20 μg | 12 | 4 · 36 ± 0 · 77 | 6 · 53 ± 0 · 97 | 8 · 67 ± 1 · 12 | 0 · 32 ± 0 · 09 |
| Oxygen + oct 10 μg bid | 6 | 4 · 97 ± 1 · 29 | 7 · 06 ± 1 · 21 | 9 · 44 ± 1 · 17 | 0 · 35 ± 0 · 15 |
| Oxygen + Woc4D 50 μg | 11 | 3 · 95 ± 0 · 66 | 6 · 13 ± 0 · 60 | 8 · 10 ± 1 · 00 | 0 · 28 ± 0 · 17 |
| Oxygen + oct 20 μg bid | 12 | 4 · 09 ± 0 · 43 | 6 · 45 ± 0 · 50 | 8 · 59 ± 0 · 94 | 0 · 30 ± 0 · 12 |
| Oxygen sham | 15 | 4 · 06 ± 0 · 83 | 6 · 29 ± 1 · 26 | 8 · 58 ± 1 · 45 | 0 · 29 ± 0 · 14 |

Values are shown as mean ± SD (g). Psac, post natal day of sacrifice; oct, octreotide.

Values are shown as mean±SD (g). Psac, post natal day of sacrifice; oct, octreotide.

What is claimed is:

1. A method of treating or preventing retinopathy of prematurity in a neonatal mammal comprising the step of administering to the mammal, an amount of a somatostatin analog containing composition sufficient to provide a therapeutic benefit.

2. The method of claim 1, wherein the somatostatin analog is octreotide, Woc4D, a somatostatin analog having a serum half-life of octreotide, a somatostatin analog having a serum half-life more than that of octreotide or a somatostatin analog having a serum half-life of Woc4D.

3. The method of claim 1, wherein the somatostatin analog is octreotide or a somatostatin analog having an ability to alter pituitary growth hormone expression similar to that of said octreotide.

4. The method of claim 1, wherein the somatostatin analog is Woc-4D or a somatostatin analog having an ability to alter pituitary growth hormone expression similar to that of said Woc-4D.

5. The method of claim 1, wherein the somatostatin analog is selected from the group consisting of octreotide, lanreotide (LANREOTIDE™), Vapreotide, Woc-2A, Woc-2B, Woc-3A, Woc-3B, Woc-4, Woc-4D, Woc-5 and Woc-8.

6. The method of claim 5, wherein said composition is administered subcutaneously.

7. The method of claim 6, wherein said mammal is a mouse or a human.

8. The method of claim 7, wherein said mammal is the human.

9. The method of claim 8, wherein the somatostatin analog is octreotide.

10. The method of claim 9, wherein octreotide containing composition is administered two or more times daily before, during, and/or after administration of supplemental oxygen to said mammal in doses sufficient to bring about suppression of retinopathy of prematurity.

11. The method of claim 8, wherein the somatostatin analog is Woc-4D.

12. The method of claim 11, wherein Woc4D containing composition is administered two or more times daily before, during, and/or after administration of supplemental oxygen to said mammal in doses sufficient to bring about suppression of retinopathy of prematurity.

13. A method of treating or preventing retinopathy of prematurity comprising the step of administering to a mammal in need thereof an amount of a composition comprising a somatostatin analog and a pharmaceutically acceptable carrier sufficient to provide a therapeutic benefit wherein the somatostatin analog is selected from the group consisting of octreotide and Woc-4D.

14. The method of claim 13, wherein the pharmaceutically acceptable carrier is a buffer soluation.

15. The method of claim 14, wherein said mammal is a mouse or a human.

16. The method of claim 15, wherein said mammal is the human.

17. The method of claim 16, wherein the selected somatostatin analog is octreotide.

18. The method of claim 17, wherein the selected somatostatin analog is Woc-4D.

19. A method of preventing retinopathy of prematurity in a neonatal human comprising the step of administering to said human, an amount of an octreotide or Woc-4D containing composition sufficient to provide a therapeutic benefit.

20. The method of claim 19, wherein the somatostatin analog is octreotide, Woc4D, a somatostatin analog having a serum half-life of octreotide, a somatostatin analog having a serum half-life more than that of octreotide or a somatostatin analog having a serum half-life of Woc4D.

21. The method of claim 19, wherein the somatostatin analog is octreotide or a somatostatin analog having an ability to alter pituitary growth hormone expression similar to that of the octreotide.

22. The method of claim 19, wherein the somatostatin analog is Woc-4D or a somatostatin analog having an ability to alter pituitary growth hormone expression similar to that of the Woc-4D.

23. The method of claim 19, wherein the somatostatin analog is selected from the group consisting of octreotide, lanreotide (LANREOTIDE™), Vapreotide, Woc-2A, Woc-2B, Woc-3A, Woc-3B, Woc-4, Woc-4D, Woc-5 and Woc-8.

24. The method of claim 23, wherein said composition is administered subcutaneously.

25. The method of claim 24, wherein the composition contains Woc-4D.

26. The method of claim 25, wherein the composition is administered one or more times daily before, during, and/or after administration of supplemental oxygen to said mammal in doses sufficient to bring about suppression of retinopathy of prematurity.

27. The method of claim 24, wherein the composition contains octreotide.

28. The method of claim 27, wherein the composition is administered two or more times daily before, during, and/or after administration of supplemental oxygen to said mammal in doses sufficient to bring about suppression of retinopathy of prematurity.

29. A method of preventing retinopathy of prematurity in a neonatal human comprising the step of subcutaneously administering to the human, an amount of an octreotide or Woc-4D containing composition sufficient to provide a therapeutic benefit.

30. The method of claim 29, wherein the somatostatin analog is octreotide, Woc4D, a somatostatin analog having a serum half-life of octreotide, a somatostatin analog having a serum half-life more than that of octreotide or a somatostatin analog having a serum half-life of Woc4D.

31. The method of claim 29, wherein the somatostatin analog is octreotide or a somatostatin analog having an ability to alter pituitary growth hormone expression similar to that of the octreotide.

32. The method of claim 29, wherein the somatostatin analog is Woc-4D or a somatostatin analog having an ability to alter pituitary growth hormone expression similar to that of the Woc-4D.

33. The method of claim 29, wherein the somatostatin analog is selected from the group consisting of octreotide, lanreotide (LANREOTIDE™), Vapreotide, Woc-2A, Woc-2B, Woc-3A, Woc-3B, Woc-4, Woc-4D, Woc-5 and Woc-8.

34. The method of claim 33, wherein said composition is administered subcutaneously.

35. A method of treating or preventing retinopathy of prematurity comprising the step of administering to a mammal in need thereof a composition comprising an amount of somatostatin analog and a pharmaceutically acceptable carrier sufficient to provide a therapeutic benefit wherein the somatostatin analog is octreotide.

36. The method of claim 35, wherein said composition is administered subcutaneously.

37. The method of claim 36, wherein said mammal is a human.

38. The method of claim 37, wherein said composition is a controlled-release composition.

39. A method of treating or preventing retinopathy of prematurity comprising the step of administering to a mammal in need thereof a composition comprising an amount of somatostatin analog and a pharmacetically acceptable carrier sufficient to provide a therapeutic benefit wherein the somatostatin analog is Woc-4D.

40. The method of claim 39, wherein said composition is administered subcutaneously.

41. The method of claim 40, wherein said mammal is a human.

42. The method of claim 41, wherein said composition is a controlled-release composition.

43. A pharmaceutical composition Woc-4D and a pharmaceutically acceptable carrier for treating or preventing retinopathy of prematurity in a neonatal mammal.
